(19) United States
(12) Patent Application Publication
(10) Pub. No.: US 2017/0042916 A1
(43) Pub. Date: Feb. 16, 2017

(54) ANIMAL TISSUE COLONIZATION AND TREATMENT OF INFECTION

(71) Applicants: Carl Hilliard, San Diego, CA (US); William R. Cast, Fort Wayne, IN (US)

(72) Inventors: Carl Hilliard, San Diego, CA (US); William R. Cast, Fort Wayne, IN (US)

(21) Appl. No.: 15/335,064

(22) Filed: Oct. 26, 2016

Related U.S. Application Data
(63) Continuation-in-part of application No. 14/716,589, filed on May 19, 2015.
(60) Provisional application No. 62/000,403, filed on May 19, 2014.

(51) Int. Cl. A61K 31/695; A61K 47/10; A61K 47/20; A61K 31/56; A61K 47/26; A61K 45/06; A61K 9/00; A61K 9/50; A61K 38/47

(52) U.S. Cl. CPC A61K 31/695; A61K 9/50; A61K 47/10; A61K 47/20; A61K 38/47; A61K 47/26; A61K 45/06; A61K 9/0014; A61K 9/0017; A61K 31/56; A61K 9/0046; C12Y 302/01004

(57) ABSTRACT

An antimicrobial composition and methods of use are provided. This antimicrobial composition includes a water-soluble organosilane (3-(trihydroxysilyl) propyldimethyloctadecyl ammonium chloride) and additional adjuvant compounds, including anti-inflammatory medications, antiseptics, transdermal penetrants, nutrients and/or buffers, generally to be applied in liquid form to penetrate and kill biofilms that are infecting living vertebrates. The methods include topically treating an infection by penetrating and disrupting an existing biofilm and killing cells therein and killing any persister cells that, through degrees of dormancy or otherwise, have escaped being killed during the initial application of the antimicrobial composition.

### CROSS REFERENCE TO RELATED APPLICATION

This application is a continuation-in-part of U.S. Utility patent application Ser. No. 14/716,589, filed May 19, 2015, which claims priority to U.S. Provisional Patent Application No. 62/000,403, filed May 19, 2014, and U.S. Provisional Patent Application No. 62/201,693, filed Aug. 6, 2015, the disclosures of which are hereby incorporated entirely herein by reference.

### BACKGROUND

Technical Field

This disclosure relates generally to antimicrobial compositions with antimicrobial activity, in particular, to organosilane quaternary ammonium compounds for topical treatment of infections caused by biofilms in ear canals, skin and sub-cutis, nails, cornea, conjunctiva, wounds and abrasions in animals and humans.

State of the Art

Prevention and treatment of infection in humans and animals has been a public health goal since the discovery of microorganisms and their role in causing disease. As an outgrowth of the germ theory of disease, much progress has been made in controlling the spread, dissemination, and effects of pathogenic microorganisms. For example, simple techniques such as routine hand-washing and thorough cleaning of hard surfaces are highly effective in preventing the spread of diseases which are disseminated by contact. When infection occurs despite such precautions, treatment with topical and systemic antimicrobials, such as the use of antibiotics, have been valuable adjuncts to these preventive measures. It is widely perceived that antiseptics and disinfectants act as general protoplasmic poisons. Antibiotics generally act by targeting a specific and vital cell function or structure; e.g. the cell wall, nuclear DNA, ribosomes or cellular enzymes. Examples are the inhibition or regulation of enzymes involved in cell wall biosynthesis, nucleic acid metabolism and repair, protein synthesis, and disruption of the membrane structure. Antibiotics that affect the structure of the cell wall act at different stages of peptidoglycan synthesis and cell wall construction.

The prevailing, but now changing, assumption has been that the evolution of single cell microbes (planktonic) has resulted in the creation of “superbugs” that are resistant to the antibiotics used in current clinical treatments. Some 700,000 people are killed each year by drug-resistant infections. The World Health Organization (WHO) has called antimicrobial resistance “so serious that it threatens the achievements of modern medicine.” Thus, the call from WHO and the Federal Drug Administration (FDA) for new antimicrobial agents and proactive measures to prevent all antibiotic and antifungal agents from being overused or leached into the environment thus promoting microbial adaption and resistance.

A concern is that there are not enough new molecules capable of being turned into antibiotic medicines. David Payne, a researcher at GlaxoSmithKline, wrote in 2015 that his firm looked at approximately 70 apparently promising targets. GlaxoSmithKline expected to find most of the molecules on the target list would be worth the effort of further study. Instead GlaxoSmithKline came away with six and, given the attrition rate for development of new drugs, considered the search as a “pretty-much wasted effort.” Economist, May 2016, Antibiotic Resistance, p. 21. The statutory definition of a new drug is “a new chemical formula or an established drug prescribed for use in a new way.” 21 U.S.C. §321(p)(2). No new class of antibiotics has been found since 1987.

It is often incorrectly assumed that populations of single cell microbes (planktonic) in a biofilm are phenotypically and physiologically identical. In fact, the embedded cells in a biofilm may be from 1,000 to 4,000 times less susceptible to the effects of drugs than were those cells in planktonic form. Less often recognized in the past, but more frequently the case in practice, it is increasingly understood that antibiotic resistance is due to the evolution of biofilms that have mutated and developed genetic changes to their cells that enable these organisms to act synergistically and in concert in order to survive an attack by an antibiotic.

A great deal has been learned about biofilms, more of a general nature than of particularity though intense research is rapidly bringing new insights. Microbial biofilms account for over 99% of microbial life. Biofilms begin as a conditioning layer of microbes and debris attached to a surface, then grow in population and diversity with exudation of a surrounding matrix of hydrated extracellular polymeric substances (EPS). They then undergo biologic organization and genetic changes, create means of chemical signaling between cells, open channels within the biofilm layer through which liquids circulate to provide nutrition for the embedded cells and egress for chemicals produced, or extruded by efflux pumps, by these cells. These channels, irregular and somewhat tortuous in form, have been measured at between 25 and 150 μM in diameter and when not excessively dehydrated, especially when in a humid or moist environment have been shown to allow a flow of liquids that permit tracking of experimentally introduced latex nanoparticles or beads (identifiable by microscopy) within the channels.

It has been confirmed that antibiotics readily enter biofilms either by diffusion (more slowly) or by channels whereby the full depth may be penetrated within minutes to hours, but not all antibiotics or germicides are alike either in their abilities to traverse the channel nor in their efficacy at different strata levels, levels where oxygen and nutrition gradients differ and where the EPS content interferes with gene expression. Some antibiotics are slowed in their progress into the biofilm (e.g. erythromycin) as are some surfactant/germicides (e.g. cetyltrimethyl ammonium bromide), while other antibiotics like ciprofloxacin move rapidly into the biofilm, however the rate of channel flow or diffusion in penetrating a biofilm is not correlated with its killing or removal efficiency. Retardation of flow into the channels of biofilms seems to be related to differences among antibiotics in reactivity to substances in the EPS matrix. It appears that penetration differences are more a function of rate than inability to penetrate.

Within a biofilm, more so when it is older and thicker, there is spatial physiologic heterogeneity with the more metabolically active microbes being in the upper reaches and cells residing in lower strata in a protective low metabolic state, some cells becoming dormant and highly resistant to treatment. Also, different antibiotics are more or less effective at different biofilm strata and against different declining levels of microbial metabolism. Mechanisms of antibiotic resistance tend to be specific for each antibiotic. Some cells within a biofilm adapt by presenting different gene expression as the biofilm is challenged with different antibiotics; this accounts for failure after a partially successful treatment when the second presentation of the same antibiotic (e.g. ciprofloxin or tobramycin) is met with altered resistance. When cells within the biofilm sense that antibiotics to which they have developed resistance are in the environment those cells utilize efflux pump system(s) in their cell walls to reduce intracellular concentrations and to prevent those antibiotics from damaging the cells from within. Antibiotics/antifungals that reach the interior of biofilm cells can be quickly expelled by an efflux pump of the individual cell under attack.

Current study about the minutiae of materials transported within the biofilm is ongoing and continuously revealing, but many aspects of biochemical interaction are incompletely understood. It is known that when the biofilm is moist or in liquid, channels within the biofilm reliably carry microbial exudation through the EPS, exudation that allows for quorum sensing (QS) between microbes and also for liquids, nutrients and oxygen to penetrate the biofilm more rapidly than by diffusion.

The establishment, maintenance and existence of biofilm communities are highly complex, socially organized processes. Approximately forty-percent (40%) of individual microbial genes change expression as they assume different functions as a result of the transition from single cell microbes (planktonic) to biofilm state. These pronounced physiological changes help biofilms to resist disinfection, to develop antibiotic resistance, to release deadly toxins and to break down materials. There may also be an exchange of genetic material (horizontal transfer) via plasmids to enable antibiotic resistance information to pass between individual microbes of differing bacterial and fungal types within the biofilm. Exchanged genetic material may confer additional resistance to an antibiotic. This process leads to the rapid establishment of antibiotic resistance within the biofilm's contiguous population of pathogens. Multi-species biofilms are a functional consortium that often possess a combined metabolic activity that is greater than the individual component species. Biofilms also may evolve by chromosomal mutations that modify the shape of the targets of antibiotics or acquire mobile elements, such as proteins or enzymes that carry genetic material (horizontal transfer) that chemically modifies or degrades antibiotics. It is these collective actions that result in antibiotic resistance.

Even less well understood is the biofilm defense of antibiotic tolerance. Because of this defense “we actually never had antibiotics capable of eradicating an infection.” Lewis (2012), Persister Cells: Molecular Mechanisms Related to Antibiotic Tolerance, p. 121. A small number of cells (persister cells) in a biofilm are phenotypically resistant to sudden exposure, to stress brought on by high doses of antibiotics and also phagocytosis by microphages. Generally, persister cells are cells that have survived initial destruction of the biofilm. Once an antibiotic concentration drops, surviving persister cells may re-establish the population, causing a relapsing chronic infection. Whether persistence and resistance represent complementary or alternative adaptions is unclear, although recent research indicates that they come from separate phenomena. Tolerance allows a population of cells to linger at the site during the decrease of antibiotic concentration which increases the probability of acquiring resistance.

The mechanism of the formation of persister cells has only recently been studied and begun to be understood. “Only a few years ago the molecular basis of persistence was still obscure. Although many genes were known to influence persister formation, they seemed so disparate and general that predicting persistence solely from genomic data would have appeared impossible.” Vogwill, et. al. (2016) J. Evol. Biol. dcl: 10.111/jeb. 12864, p. 1. “The main focus of research in antimicrobials has been on drug resistance, and the basic starting experiment is to test a clinical isolate for its ability to grow in the presence of elevated levels of different antibiotics.” Persister cells are missed by this test. Lewis (2012), Persister Cells, p. 124.

There are several models to explain how persister cells escape destruction. Some theories postulate that survival of persister cells may be due to genetic adaptation resulting from quorum sensing or extracellular DNA within the EPS matrix, or that persister cells adopt, at times of stress, a low metabolic state or dormancy and thus become highly resistant to antibiotics. An experiment with ciprofloxacin indicated that a biofilm response was the stress release of the TisB peptide which binds to the membrane of the persister cell causing a shutdown, blocking antibiotic targets, and ensures multidrug tolerance for the surviving persister cells.

In some instances, the medications available for treatment are limited because the available antibiotics do not have significant activity across groups of potential pathogens, and there is a lack of potent antibiotic and fungicidal agents. For example, there is question about “whether the otitis is curable, and whether treatment must be long term for resolution or that lifelong management will be required.” (Merck Vet Manual, eye and ear/otitis externa.) “[A]ntimicrobial drugs that specifically target biofilm-associated infections are needed.” CDC, Vol 10, Number 1” Fungal Biofilms and Drug Resistance.” It is apparent that there is a critical need to find and identify compositions that can overcome both antibiotic resistance and antibiotic tolerance and can destroy biofilms and persister cells.

### DISCLOSURE OF EMBODIMENTS OF THE INVENTION

The present disclosure relates generally to new and novel antimicrobial compositions and their use, in particular, to organosilane quaternary ammonium compositions and their use, to interfere with the communication between biofilm cells within the biofilm matrix, in order to treat infections of ear canals, skin, nail beds, cornea, conjunctiva and/or subcutaneous tissues in animals and humans and to prevent biofilm re-colonization of wounds and tissues by persister cells. The active ingredient in these compositions is 3-(trihydroxysilyl) propyldimethyloctadecyl ammonium chloride (hereinafter also referred to as “Trihydroxy”), a pesticide which has not yet been approved for use by the FDA to treat infections in humans or animals. Antibiotics are most frequently poisons that kill after penetrating into the interior of individual cells, or are consumed by reacting with cell walls during the killing process, whereas Trihydroxy works from outside the cell and is not subject to efflux pump resistance from inside the cell that expel antibiotics and antifungals. Trihydroxy is not consumed by chemically reacting with microbes in bringing about their death. Other existing drugs used to treat ear and skin infections attempt to coat or cling to tissues to keep the medication in place. The use of such thick and/or sticky carriers make such products far less able to penetrate a stenotic ear canal, a feature common to the most difficult to treat infections involving scar or edema. The disclosed compositions include aqueous solutions.

The disclosed compositions establish bonds to the infected tissue that last through the skin replacement cycle to kill any persister cells that may survive the initial treatment, later to reactivate. Further, antibiotics and antifungals lose strength during the treatment process, with ever declining levels of concentration, which enables persister cells to survive and to develop defenses to the pharmacological profile of the antibiotic. The next time an antibiotic is needed, it may not be effective to treat what was once a more easily treatable infection. In addition, antibiotics and antifungals each have specific and limited ranges of effectiveness against either Gram positive bacteria, Gram negative bacteria, yeasts and/or fungi, whereas Trihydroxy exhibits a wide spectrum of activity against Gram positive bacteria, Gram negative bacteria, fungi and yeasts. Trihydroxy's broad killing range reduces microbial exposure to multiple antibiotics, thus reducing emergence of widespread antibiotic resistance. Polypharmacy and the repeated and routine exposure of antibiotics to farm animals has been an important cause of generalized antibiotic resistance. For this reason, a ban on use of antibiotics in animal feed without veterinary prescription is now being considered.

The molecular size of the Trihydroxy is about 28 to 40 nM in height and about 10 nM in width, small enough to traverse the biofilm channels. The channels, being necessary for biofilm functions that include quorum sensing (QS), permit the organosilane quaternary ammonium compounds to flow along with nutrients within the channels. The positive charge through the presence of the N+(nitrogen) atom of the Trihydroxy molecule is important in aiding flow and in providing ionic attraction to the negative cell walls. It is well known that molecular diffusion occurs in biofilms, but diffusion is far slower than transfer by flow through channels. A research article in 1996 described antibiotic penetration of biofilms predicting that their marked loss of effectiveness was not adequately accounted for by diffusion or by differing gradients of pH or oxygen in layers of the biofilm.

Quaternary ammonium compounds are widely used for industrial control of bacterial growth, and their effectiveness has persisted since their introduction in the 1930's. (There are many hundreds of commercial products that contain different quaternary ammonium compounds (QACs.) Conventional uses include disinfectants, surfactants, fabric softeners, antistatic agents, and/or wood preservers. Each quaternary ammonium compound has its own chemical and anti-microbiological characteristics. These QACs are commonly used in pesticides and/or cleaning agents that typically have formulations that include enzymes, adjuvants and other disinfectants that are used in high concentrations; they are often toxic and harmful to the skin and eyes.

An antimicrobial pesticide is generally defined as a pesticide that is intended to disinfect, sanitize, reduce or mitigate growth or development of microbiological organisms on inanimate objects (7 U.S.C. §136 (mm)). Some of these pesticide and cleaning agents establish a permanently bonded covering, covalently bonded to a surface, such as a wall or countertop. These pesticides become effective only upon drying and are not intended for use as aqueous solutions. The dried covering reduces the number of surviving planktonic microorganisms by killing some, but not all, that come into contact with the treated surface. These compositions tend to be self-deactivating via “protein loading,” when the surface becomes overburdened with debris. There is no known or certified use of these pesticides for treating infections on and/or in animals and humans.

Oxygen gradients and nutrient concentrations are lowest in the deepest layers of the biofilm, conditions thought to be among the most critical stresses in promoting the formation of persister cells. Channels allow flow of liquids to reach the lowest layers of the biofilm and would permit molecules the size of Trihydroxy to reach persister cells in the base layer. Within the channels, it is thought that the positive charge of the nitrogen atom attracts the negatively charged cell wall and by affecting the phospholipids and proteins of the cell wall, making the cell wall more permeable; the organosilane's carbon chains then contact, penetrate and entangle cell walls resulting in the permanent loss of a cell's reproductive and metabolic activity. This takes place while Trihydroxy is in solution and also when Trihydroxy is bonded to tissue. The killing action of the compound is thought to be the same for active and persister cells as long as the target cell has a negative charge on its cell membrane. Thus, by first being active in the upper layer of the biofilm, the composition with treatment protocol of several days, can reduce the population of cells and the thickness of the biofilm. This makes the lowest level more accessible and is likely to create improved nutritional and oxygen gradients promoting an increase in the metabolic activity of the cells located at the base. This activity, unlike that of many antibiotics that are consumed by their killing action, would not necessarily be deactivated after killing a single cell, and with collapse of a cell, the Trihydroxy may remain in channel flow or in the EPS of the film to kill cells in lower layers.

This composition has the additional benefit of binding to the surface of the tissue treated, thereby remaining effective for the approximately 28 days before skin is sloughed or replaced. This allows for potential destruction of any persister cells that survive the initial attack. The topical application of the composition may be repeated at a dose and frequency effective to treat the pathogenic microbial infections having a negative cell wall charge that is ionically attracted to a positive charge of the composition and pulled into a projecting chain of carbon atoms that disrupt and breach cell walls causing microbial cell death.

The disclosed antimicrobial treatment methods use an organosilane, a carrier, and a delivery system. The disclosed methods treat infections caused by pathogens that have invaded human and/or animal tissues of the ear, layers of the skin, nail matrix, nail bed, cornea and/or conjunctivae and that have multiplied, forming biofilms causing disease. The methods comprise flushing onto infected tissue an aqueous antimicrobial composition comprising an organosilane quaternary ammonium compound, killing persister cells and biofilm and establishing a declining periodicity of use, until there are days or weeks between applications of the composition, which is effective to treat both the acute phase of infection and also reactivation of any remaining persister cells that can, by reactivation, cause relentlessly recurring, chronic infections. Treatment of skin, ear, cornea, conjunctiva and nail bed infections is likely to require repeat applications. As the tissue infection is reduced, as access becomes eased with decreased swelling, and as the biofilm is destroyed, more of the composition becomes bonded to the tissue. The residual, immobilized Trihydroxy will continue to treat the infection until the tissue sloughs or the skin is shed. The composition is of low toxicity and will not promote resistance to antibiotics, having an environmental profile showing toxicity only to fish. As shown in the following examples, three canines with serious otitis infections that did not respond to traditional treatments with heavy MICs of antibiotics were cured by the disclosed composition and became infection free.

The compositions disclosed herein are intended, inter alia, to cure otitis externa and serious skin infections and to prevent complications leading to canal stenosis, hearing loss, cellulitis, abscess formation or septicemia. The skin is the largest organ of the body. The adult skin weighs about 8 pounds with an area of about 22 square feet. Skin acts to sense stimuli, to regulate temperature and to protect the body. However, skin can also become infected. Each area of the skin is different in hair bearing, in color, in oil glands, in perspiration, in blood flow and in healing and resistance to infection. The scalp, perirectal and facial areas tend to heal well and resist infection, while lower extremities tend to heal more slowly. The skin of the ear canal in dogs, for example, can become a reservoir of infection and a fomite to the companion animal's household. Biofilm infections are resistant to treatment and can support a mixed population of opportunistic pathogens that lead to cellulitis, abscess formation and life threatening septicemia. Bacterial skin infections are a major problem for older people and people with chronic health conditions, such as diabetes. Infected wounds heal more slowly, causing pain and distress to the patient, and are expensive to treat. Because of its quantitative and qualitative importance, effective treatments of infections of the skin are crucial not only for the well-being but for the very survival of humans and animals. The compositions disclosed herein provide an effective alternative to existing medications that are not effective or that are losing effectiveness. The compositions bring long term relief from recurrence and promise to improve all aspects of clinical treatment for skin, ear, conjunctiva and nail bed disease caused by bacterial or fungal biofilm infections. The disclosed compositions are not antibiotics, and they lack the disadvantages of promoting antibiotic resistance and tolerance.

In some embodiments, the delivery system is a microcapsule enclosing the Trihydroxy molecule or composition therein and/or a microcapsule coated with the Trihydroxy molecule or composition for treatment of deep wounds, lacerations or infections deep in the ear canal. In some embodiments, the concentration of the Trihydroxy is less than about 0.10 percent by weight. In some embodiments, the concentration of the Trihydroxy is between about 0.10 percent and about 1.00 percent by weight. In some embodiments, the concentration of the Trihydroxy is greater than about 1.00 percent by weight. In some embodiments, the concentration of the Trihydroxy is greater than about 5.0 percent by weight. In some embodiments, the concentration of the Trihydroxy is greater than about 10.0 percent by weight. In some embodiments, the carrier is selected from the group consisting of water, an alcohol, propylene glycol, a petroleum-based ointment and/or mixtures thereof. In some embodiments, the concentration of Trihydroxy is in the range of from about 0.01 w/v % to about 25.0 w/v % in water. In some embodiments, 3-(trimethoxysilyl) propyldimethyloctadecyl ammonium chloride is mixed into an aqueous solution which hydrolyses to form 3-(trihydroxysilyl)propyldimethyloctadecyl ammonium chloride, i.e. the 3-(trihydroxysilyl) propyldimethyloctadecyl ammonium chloride is formed by reaction of 3-(trimethoxysilyl) propyldimethyloctadecyl ammonium chloride and water.

In some embodiments, the composition further comprises an enzyme. In some embodiments, the enzyme is a proteolytic hydrolase enzyme, a protease. In some embodiments, the enzyme is an enzyme acting upon a substrate comprising N-acyl homoserine lactone to temporarily compromise the biofilm signaling system. In some embodiments, the enzyme is a cellulase. In some embodiments the enzyme is a lipase.

In some embodiments, the composition further comprises an anti-inflammatory. In some embodiments, the anti-inflammatory comprises a steroid. In some embodiments, the anti-inflammatory is selected from the group consisting of hydrocortisone, triamcinolone diacetate, beta methasone valerate, beta methasone diproprionate, hydrocortisone and/or mixtures thereof. In some embodiments, the composition further comprises an antiseptic. In some embodiments, the antiseptic is selected from the group consisting of-hexylresorcinol, methyl resorcinol, ethyl alcohol and/or mixtures thereof. In some embodiments, the composition further comprises a penetrant to aid in treating infections in or covered by a thick layer of keratin, such as nail bed keratin. In some embodiments the penetrant is dimethylsulfoxide (DMSO).

In some embodiments the composition comprises a buffer selected from the group of consisting of a citrate, a sulfonate, a carbonate, a phosphate and/or mixtures thereof. In some embodiments the composition contains a nutrient, such as a salt or simple sugar solution to adjust tonicity, to delay physiological changes in the biofilm and/or to effect nutritional improvement in the biofilm, in particular to delay persister cell dormancy and to aid in awakening dormant persister cells.

In some embodiments, the method further comprises a step of penetrating a biofilm. In some embodiments, the method further comprises a step of placing treated material in proximity to an area of microbial biofilm presence.

The foregoing and other features and advantages of the present invention will be apparent from the following more detailed description of the particular embodiments of the invention.

### BRIEF DESCRIPTION OF THE DRAWINGS

Some of the embodiments will be described in detail, with reference to the following figures, wherein like designations denote like members:

FIG. 1 is a schematic diagram showing a general chemical structure of an organosilane molecule according to the invention; and.

FIG. 2 is a schematic diagram showing a general chemical structure of an organosilane molecule according to the invention.

### DETAILED DESCRIPTION OF EMBODIMENTS OF THE INVENTION

A detailed description of the hereinafter described embodiments of the disclosed method are presented by way of example and not meant to be limiting with reference to the Figures listed above. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise. Some general definitions are provided for the terms used herein. “Biofilm” is any population of microbes living in organized structures that possess remarkable structural complexity that infect tissue of living vertebrates. These cells are commonly embedded within a self-produced matrix of extracellular polymeric substance (EPS). Microbes in this EPS state make collective “decisions (biochemical adjustments)” in that genetic changes are made among the cells by communicating with chemical signals known as quorum sensing. The cells growing in a biofilm are physiologically distinct from planktonic cells, which, by contrast, are single-cells that may float or swim in a liquid medium. “Microbial cell” and “microbe” are used interchangeably and are understood to mean any single-celled planktonic micro-organism, bacteria, yeast and/or fungus. “Microcapsule” refers to a subset of the broader category of “microparticles,” wherein the microcapsule is a microparticle having a core comprising one material, compound or composition surrounded by a distinctly different second material, compound or composition. As the generally accepted size range for microparticles, a microcapsule has a size within the broad range of from about 1 micron to about 1000 microns (1 millimeter). Therefore, the size range of a microcapsule, for the purposes of this application, is between that of a large nanoparticle to an object visible to the eye without magnification. Microparticle may also refer to a solid compound comprising the particle that is, itself, coated with the organosilane quaternary ammonium compound for purposes of becoming imbedded in a conditioning layer or more mature biofilm.

Disclosed herein is a method for treating pathogenic microbial infections of animals and humans, the method comprising: topically applying to wounds and/or infected tissue a composition comprising a liquid preparation comprising 3-(trihydroxysilyl) propyldimethyloctadecyl ammonium chloride at a concentration in a range of from about 0.01 w/v % to about 25.0 w/v % in water; and repeating the topical application of the composition at a dose and frequency effective to treat the pathogenic microbial infections having a negative cell wall charge, wherein the negative cell wall charge is ionically attracted to a positive charge of the composition causing microbial cell death.

The pathogenic microbial infections may be of skin, subcutaneous tissues, cornea and/or conjunctiva. The 3-(trihydroxysilyl) propyldimethyloctadecyl ammonium chloride may be formed by reaction of 3-(trimethoxysilyl) propyldimethyloctadecyl ammonium chloride and water. The liquid preparation may further comprise propylene glycol. The composition may further comprise dimethylsulfoxide. The composition may be encapsulated within microcapsules for a delayed dose. The composition may also be coated on an outer surface of microcapsules for an immediate dose. The composition may be encapsulated within microcapsules and coated on an outer surface of the microcapsules to provide for an immediate dose and a delayed dose of the composition. The wounds and/or infected tissue may comprise a biofilm having a biofilm matrix. The composition may further comprise an adjuvant comprising a proteolytic hydrolase enzyme. The enzyme may be N-acyl homoserine lactone. The enzyme may be a cellulase.

The composition may further comprise a nutrient. The nutrient may be a sugar. The sugar may be selected from the group consisting of monosaccharides, disaccharides, polyols and/or mixtures thereof. The sugar may be a monosaccharide. The monosaccharide may be dextrose. The composition further comprises an anti-inflammatory. The anti-inflammatory may be a steroid. The-anti-inflammatory may be selected from the group consisting of hydrocortisone, betamethasone, dexamethasone and/or mixtures thereof.

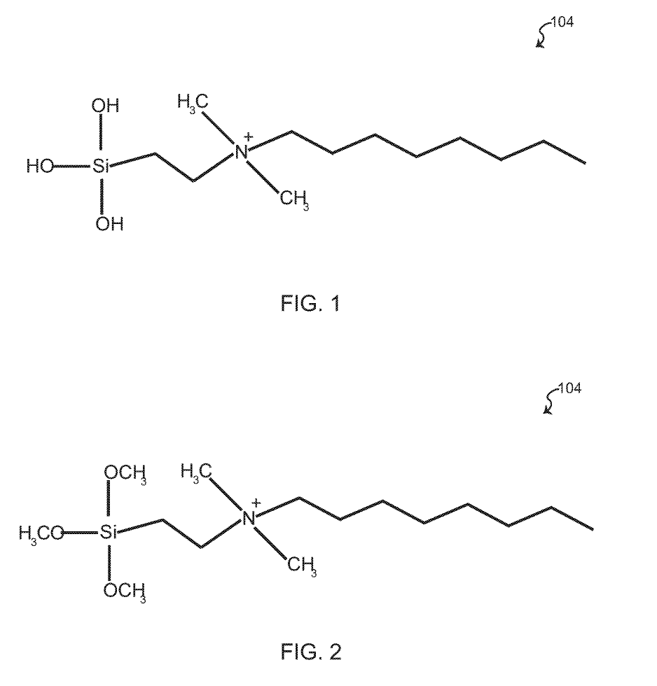

Trihydroxy 102 is illustrated in FIG. 1. The antimicrobial composition according to the invention comprises Trihydroxy 102 alone or in combination with other compounds in a mixture chosen according to the intended application of the composition. The antimicrobial activity of the composition is provided by the Trihydroxy 102. The Trihydroxy comprises silicon covalently bonded to carbon. Trihydroxy 102 comprises a hydrophilic “cap” comprising a silicon-tri-methoxy or silicon-tri-hydroxy “head,” and a hydrophobic “tail” comprising an eighteen or twenty-atom linear carbon chain. The head and tail are joined at a nitrogen atom bonded with two additional methyl groups to create a (cationic) quaternary ammonium group. The Trihydroxy 102 head groups facilitate chemically binding the organosilane to a human or animal tissue.

Bacterial and fungal cells in a biofilm generally carry a negative net charge on the cellular wall due to constituent membrane lipid proteins, and the positively-charged hydrophilic quaternary ammonium group allows for ionic attraction to those negatively-charged cell walls. It is believed that once being attracted to the negative cell-wall charge, the ionic forces having weakened the cell wall, the linear hydrophobic hydrocarbon tail of the organosilane engages, entangles or penetrates the already damaged phospholipid cell membrane, disrupting the membrane, causing lysis with death of the cell. Since Trihydroxy is amphiphilic, the hydrophilic portion of the molecule may traverse both the bacterial cell wall and cytoplasmic membrane, causing cellular lysis and death of the cell. As a result, the attraction and joining of the composition comprising Trihydroxy to living tissue results in such tissue becoming configured to kill reactivated persister cells. This method of killing microbes is effective while in the liquid medium. This microbial killing mechanism is advantageous for several reasons. Trihydroxy 102 is not altered or consumed by its interaction with the targeted microbe, and the method of killing cells occurs in the liquid or gel medium. Residual Trihydroxy may bind to tissue and remain until sloughing of the tissue in a normal skin cycle a feature that further limits environmental contamination. The Trihydroxy is of very low toxicity and will minimally impact the environment.

In various embodiments of the invention, other compounds may be added to the composition. For some embodiments an anti-inflammatory is added to reduce inflammation and itching. In some embodiments, the composition further comprises other enzymes or compounds to aid in penetrating the EPS matrix of the biofilm. In some embodiments, the composition comprises an agent to modify viscosity, such as propylene glycol. In some embodiments, the composition comprises an agent to promote trans-epithelial delivery through skin and keratin. In some embodiments, dimethylsulfoxide is added to help penetrate the keratin of nails.

In some embodiments a nutrient is added. The addition of nutrients, such as sugars, effectively feeds the biofilm promoting delay in persister cell formation leading to dormancy and encourages persister activation thus making the active microbe easier to kill by an organosilane that benefits from a strong negative charge on the microbial cell wall. Sugars include, but are not limited to, monosaccharides, disaccharides and polyols. Monosaccharides, such as dextrose, are particularly useful. In some embodiments dextrose or other sugars may be used with normal saline or Ringer's solution.

3-(trimethoxysilyl) propyldimethyloctadecyl ammonium chloride (hereinafter also referred to as “Trimethoxy”) 104, is illustrated in FIG. 2 only as a precursor to the Trihydroxy molecule, when hydrolyzed by addition to water, shown in FIG. 1. Trimethoxy 104 is unstable in water and is often transported in about 40% to about 50% methanol. Conventional methods of use of Trimethoxy 104 includes addition of a dilute solution in methanol to water. The EPA label required for Trimethoxy compounds states: “Danger. Corrosive. Causes irreversible eye damage and skin burns. Methanol may cause blindness. May be fatal if inhaled. May be harmful if swallowed or absorbed through the skin.”

The result of hydrolysis of 3-(trimethoxysilyl) propyldimethyloctadecyl ammonium chloride is {RSi(OMe)3+3H2O-→3 MeOH+RSi(OH)3} and the formation of 3-(trihydroxysilyl) propyldimethyloctadecyl ammonium chloride, a molecule that is stable with no other additives in water. The compound must then be used within a short period of time, such as a few hours to at most 12 hours, to treat a surface or fabric to produce a permanent surface coating.

The effectiveness of Trimethoxy or Trihydroxy when used as a pesticide on inanimate articles depends on bonding, to a cleaned and often sanitized surface, and drying. During the drying process, the molecules condense on the surface and create a permanent bond. By permanently altering the surface it is applied to, the silane monomer crosslinks to itself, polymerizes and resists microbial colonization by reducing the number of microbes that randomly land on the treated surface. This static or “immobilized” pesticide does not eliminate all of the planktonic microbes that contact an inanimate surface and requires that the delivery system involve drying and then bonding in order to become effective. The surface protection created by these bonded molecules depends on regular cleaning since the barrier is only about 20 to about 40 nM in depth and is made ineffective when overburdened by micro-detritus and debris. Bacteria generally range in size from about 120 nM to about 1000 nM and so coverage of the barrier by detritus from killed microbes makes the covering ineffective. In fact, detritus and debris may form an attractive conditioning layer, a precursor to a biofilm bed for planktonic microbes, if not regularly cleaned.

The killing ability of the composition according to the invention results from the Trihydroxy molecule and not the carrier or any added adjuvants. The composition targets established infections of the skin, subcutaneous tissues, ears and/or conjunctiva. The composition in liquid form is directly applied to the infected tissue, without drying. Trihydroxy will be a first in class drug when approved by the FDA for treatment of an established infection.

Embodiments of the composition according to the invention comprise Trihydroxy, and in some embodiments, additional structural and functional components, adjuvants that complement one another to add functionality and performance to the composition, the structure and function of which will be described in greater detail herein.

In some embodiments involving treatment with the composition of an infected tissue, inflammation and edema are present. An anti-inflammatory compound is a useful therapeutic adjunct to reduce swelling, to gain additional access in the case of a narrowed ear canal and to reduce trauma from itching and scratching. Biofilm infection normally creates an inflammatory response. Inflammation creates local swelling, increases pain and/or itching, and, if marked, may interfere with healing. This is particularly important in veterinary application wherein inflammation compels the animal to dig or chew at the infected tissue substrate, aggravating inflammation and leading to a cycle of increased irritation and more scratching. Skin and ear canal infections are so common as to be a leading cause of visits to a veterinarian, too often after weeks of home treatment have failed.

Skin infections are also a major problem for older people and people with chronic health conditions, such as diabetes, and contact with infected companion animals is an increased risk. Infected wounds heal more slowly, causing pain and distress. Incidence numbers of these infections are so large as to be a serious problem if only on economic grounds. Difficult cases however are not rare, especially for example, in companion animals with allergies, eczema, or diabetes; such disease is serious in degrading life-style through pain, itching and wasting of general health. In particular, with repeated occurrence, a common problem for several breeds, this morbidity has substantial negative effect on day to day functioning, including itching, pain, stenosis of ear canals and hearing loss. In veterinary medicine, otitis externa is a serious problem particularly with biofilm formation and in dogs that may require ablative surgery for stenosis of ear canals. Also, in the case of lacerations, particularly those that may expose tendons, the resulting tendinitis can lead to loss of a limb or loss of the animal's life.

Treatment with a topical or systemic anti-inflammatory compound is often useful. In some embodiments, the composition further comprises an anti-inflammatory. Some non-limiting examples of anti-inflammatory compounds include steroids, such as triamcinolone diacetate, hydrocortisone, beta methasone valerate, and beta methasone diproprionate. In some embodiments, the composition further comprises a topical anesthetic to treat the pain and itching associated with the inflammatory response, some non-limiting examples including benzocaine, lidocaine hydrochloride, hexylresorcinol and methyl resorcinol.

Chronic ear infections result in a cycle of inflammation, infection and thickening of the tissue lining the ear canal, which eventually leads to narrowing of the ear canals (stenosis) preventing traditional medications from reaching the infected portions of the canal. Ulcerations of the ear canal can also result from infection or self-trauma caused by scratching. Even in mild to moderate incarnations, otitis infections involve pain, loss of hearing and suffering. The occluded canals also prevent the natural sloughing the canal's skin cells, sebum, and hair, which accumulates in both the canal and possibly the middle ear, thereby making the infection more dangerous. Otitis becomes a serious and life threatening disease when it causes stenosis, abscess formation, when it exposes bone and periosteum to invasive and resistant biofilm infection, and when through membrane perforation it then involves the middle ear and mastoid cavities. Progression of the infection into the middle and inner ear can cause severe complications, including a head tilt, incoordination, inability to stand or walk, hearing loss, and unrelenting pain. “Methicillin-resistant Staphylococcus Aureus and Pseudomonas otitis (caused by Pseudomonas aeruginosa) have emerged as frustrating and recurring causes of otitis because of the development of resistance to most common antibiotics.” And “resistance is developing to fluoroquinolones.” Merck Vet Manual, eye and ear/otitis externa.

Pre-clinical tests show that the composition cures chronic otitis and prevents re-infection from biofilms. Evidence from tests of the composition has demonstrated substantial improvement over existing therapies with regard to effectiveness, cost, frequency of use, and reduction of chronicity due to biofilms and persister cells. Thus, the composition serves an unmet medical need to cure episodes of relentlessly recurring chronic otitis due to biofilms, and thus provides pain-free and easy lifetime management of this previously termed “incurable” disease, even should it reoccur through repeated water contamination or other introduction of new pathogens into the ear or skin.

One of the advantages of the described composition includes no promotion of persister cell tolerance (because the described composition kills persister cells) effectively disabling and destroying biofilms. Other advantages include, but are not limited to, pattern of use (twice daily or once daily); ease of compliance; low toxicity; treatment of conditions that are intolerant to or allergic to previously tried antibiotics; absent cross reactivity with antibiotics; bonding to tissue until sloughed and thus lingering in the treated area acting as treatment if persister cells reactivate; efficacy against treating yeast, fungus and bacterial infections with a single agent thus allowing initiation of effective treatment prior to results of cultures becoming available; providing nutrients to activate persister cells; and cost savings based on the cost of the organosilane quaternary ammonium compound as compared to the cost of antibiotics or combined antifungal agents.

Some antibiotics and enzymes function optimally within a relatively narrow pH range. Accordingly, in some embodiments, a buffer may be added to the composition at concentration levels sufficient to maintain the pH range required for optimal activity of the described composition. The particular buffer is selected based upon the local conditions present on the biological substrate. Buffers to maintain ambient pH within a desired range include, but are not limited to, citrates, sulfonates, carbonates, and phosphates. The preferred buffering compound and concentration of same useful for maintaining a desired pH range are dependent on ambient micro-environmental conditions at the treated area and known to those skilled in the art.

Biofilm cells may include bacteria, archaebacteria, protists, and/or fungi, and in particular may include cells that have survived initial destruction of the biofilm, the persister cells. Persister cells become dormant as they live in lower strata of the biofilm that have gradients of lower oxygen and nutrition than strata above; this gradually causes the microbe to reduce metabolic activity leading eventually to dormancy and extreme resistance to being killed by antibiotics and antimicrobials. As long as there is a negative cell wall charge on the microbial cell wall, Trihydroxy can function to kill the microbe. The addition of a nutrient in some embodiments of the composition encourages persister cells to stay active or reactivate. Should the cell wall charge be reduced or absent through dormancy, the cell will reactivate when its environment is propitious, usually thought to be when more oxygen and nutrients are present. The method of treatment herein described kills reactivated persister cells and thus reduces the chronicity of infections.

In some embodiments, the composition comprises a carrier. This carrier is a compound that holds the various sub-components of the composition in suspension or solution. The specific compound used is chosen based upon the characteristics necessary for the end-use application of the composition. For example, if the composition is to be used on a skin lining the external auditory canal of a dog, the carrier may be water, an emollient, wax, alcohol, non-ionic surfactant, and/or other suitable compound. Non-limiting examples include excipients such as cetyl alcohol, tyloxapol, methyl paraben, polyethylene glycol, coconut oil, or cottonseed oil. The carrier is, in some embodiments, may be employed to form the composition into a gel, lotion, ointment, liquid solution, or liquid suspension, according to the intended end-use of the composition.

The concentration of Trihydroxy by weight of the composition is also selected according to the desired end-use of the composition. In situations where high antimicrobial activity is needed for treating a well-established biofilm, concentrations of Trihydroxy provide a higher density of adherent molecules at the site of the infection. Additionally, higher Trihydroxy concentrations create a higher cationic charge density, resulting in both stronger electrostatic microbial attractive forces and detergent effects on the microbial phospholipid cell membrane. Concentrations of Trihydroxy in the composition of up to and over about 5% by weight may be used, however, when used in concentrations of over about 3%, polymerization of Trihydroxy within the composition prior to application increases through intermolecular cross-linking via —S—O—S— covalent bonds. In applications to deep seated infections, such as a cutaneous epithelium or an open wound treated using the composition, composition shedding through epithelial turnover may requires frequent re-application of the composition, in some applications. The risk of bacterial and other microbial resistance to an antimicrobial compound, regardless of the mechanism of action of the compound, theoretically increases with increasing environmental encounters between bacteria and other microbes, and the antimicrobial compound. It is prudent, therefore, to strive to minimize the amount of any composition with antimicrobial activity within the general environment. Accordingly, in the aforementioned and other situations wherein frequent re-application of the composition is necessary, lower concentrations of Trihydroxy, down to and below about 0.1% by weight in the composition, are useful by lowering the overall amount of Trihydroxy ultimately discharged into the environment. Notwithstanding the theory, it is believed that the risk to the environment by use of these formulations is minimal.

Some embodiments of the composition further comprise proteolytic and other enzymes as components useful in disrupting an established biofilm. Expansion of colonies of biofilms on substrates, such as the skin lining the external ear canal of a human or other animal, is enhanced by the process of constant desquamation. Biofilms in this and other examples are disrupted by incorporation of enzymatic adjuncts, such as any one of the keratinase family of proteolytic enzymes. A few non-limiting examples include incorporation of collagenase, cellulase, or keratinase into the composition. In addition to proteolytic keratinases, some embodiments of the composition comprise other enzymes. For example, N-acyl homoserine lactone is a bacterially-produced amino sugar acting as a hormone involved in quorum sensing. Some actions of N-acyl homoserine lactone include bacterial self-limitation of microbial population density and other population-based characteristics, such as gene regulation of enzyme systems and the expression of flagella versus pili. Enzymes acting upon an N-acyl homoserine lactone substrate destroy and substrate and thereby disrupt bacterial signaling systems in a biofilm, acting as an adjunct to proteolytic keratinases and other components of the composition, in some embodiments, to disperse existing biofilms and interfere with new biofilm formation.

In one embodiment, microcapsules are used as the delivery system for the composition. Microcapsules, in some embodiments, comprise a material enveloping and containing the composition. Non-limiting examples of compounds used to form microcapsules include polyvinyl alcohol, cellulose acetate phthalate, gelatin, ethyl cellulose, glyceryl monostearate, bees' wax, stearyl alcohol, and styrene maleic anhydride. Many other compounds may be used to form microcapsules, and the exact composition, construction, and manufacture of a microcapsule is chosen from a broad range of compositions and manufacturing techniques for microcapsules generally, and which are readily available and known to those skilled in the art. The liquid composition may be encapsulated within microcapsules and thereafter released when the microcapsules are broken. Breakage of a microcapsule is effected at a chosen time and in a manner specific to the particular use of the composition. Microcapsules may also be coated on the external (outer) surface with the liquid composition and/or encapsulated and coated with the liquid composition, providing an immediate dose (coated) and a delayed dose (encapsulated) of the composition. For example, a microcapsule may be broken by scratching, as when a dog scratches the ostium of its external auditory canal in response to itching arising from inflammation. Because the composition becomes active upon breaking of the microcapsule, the effective useful life of the composition begins.

The following examples are descriptive and not meant to be limiting.

### Example 1

A formulation of the disclosed composition was successfully used to cure a severe long-standing case of a mixed bacterial/fungal infectious otitis externa and infectious dermatitis in a neutered male Shih Tzu dog (Conner), aged 11 years 7 months, presented for re-evaluation of bacterial and monilial dermatitis (Malassezia sp.). Conner had a past history of keratoconjunctivitis sicca, generalized demodicosis, and allergies. The evaluation and subsequent treatment with the disclosed composition began two months after failed conventional therapy with oral antibiotics (amoxicillin/clavulanate (Clavamox® 13.6 mg/kg) orally b.i.d. for four weeks) and an oral antifungal (fluconazole, 5.4 mg/kg once daily for two weeks; then once every-other-day for two additional weeks.) The earlier failed therapy consisted of bathing the animal 2-3 times a week using an anti-seborrheic shampoo (KeratoLux®) and an antimicrobial shampoo (Duoxo Chlorhexidine® shampoo) followed by an oatmeal-based cream rinse (Episooth®). Conner's chest, neck, paws, and face were cleaned and treated twice daily with antimicrobial wipes (Douxo Chlorhexiding pads®) and an antimicrobial lotion (ResiKetoChlor®). The ears were cleaned once daily with a tris-EDTA/ketoconazole solution (TrizUltra plus Keto®) and treated twice daily with an amikacin otic preparation.

Dermatologic examination revealed extremely abundant purulent exudate in both ears with stenotic canals and erythema, lichenification, and edema on both medial pinnae. The animal had generalized mixed hypotrichosis/alopecia, erythema, hyperpigmentation, and lichenification with crusting over dorsal trunk, legs, paws, and ventrum. Hair on the face and neck was severely matted. The skin underlying the mats was crusted, with moist dermatitis and brown purulent exudate. Lymph node enlargement was palpated in the mandibular, prescapular, and popliteal node groups.

Cytological examination of skin scrapings revealed abundant bacterial dermatitis with cocci and diplococci. A generalized severe Malassezia (yeast) dermatitis was concentrated mostly on ventrum and paws. Otic examination revealed bilateral severe bacterial otitis externa with cytological examination revealing abundant mixed population of rods and cocci, along with occasional Malassezia. Demodex canis was seen in all life stages. Fine-needle aspirates of peripheral lymph nodes (left prescapular and left popliteal) were consistent with reactive lymphadenopathy.

The otitis externa was treated initially by cleaning the ears with a salicylic acid based ear cleaner (Otoclean®) and instillation of 0.5 ml of the disclosed composition comprising Trihydroxy in each ear. Mats were removed and the dog was bathed and groomed over two days using (Splash plus® shampoo), followed by antimicrobial shampoo (Douxo Chlorhexidine® shampoo), followed by essential fatty acid cream rinse (Hylyt® cream rinse). After bathing, the composition comprising Trihydroxy was applied to facial folds and over body using moistened gauze sponge pads.

Conner returned 7 days later for a brief recheck. Otic cytology revealed complete resolution of the bilateral bacterial otitis externa and significant improvement of the bacterial dermatitis at all sites. Only a mild amount of ceruminous exudate was observed in each ear. Lichenification and moist dermatitis decreased substantially. No purulent exudate was observed at any site. Topical therapy was repeated (bathing with Splash plus® shampoo, Douxo Chlorhexidine® shampoo, and Hylyt® cream rinse) followed by application of the composition solution using moistened gauze pads. Otic therapy was repeated with a cleaning using salicylic acid based ear cleaner (Otoclean®) and treated by instilling composition in each ear. Weekly rechecks over the course of one month showed continued improvement and no recurrence of bacterial or Malassezia infections. By way of clinical observation, Conner, a dog previously highly resistant to treatment, remained infection free using drops only in each ear every few weeks.

### Example 2

A fawn Puggle (Lillie) born 4/2009, was seen for an acute episode of otitis in her left ear in November 2014 after no previous history of any trauma, bathing, swimming, or medical issues. A purulent discharge was exuding from the ear canal and inflammation was apparent in the aural pinna on both the anterior and posterior aspect. A culture swab was collected for Clinpath® ID and sensitivity and a solution of the composition with steroid was directed to be flushed daily into the ear canal after the ears were cleaned utilizing a Q-tip soaked in a general cleansing solution.

Results of the culture and sensitivity showed a mixed infection of Pasteurella Multocida 1+, coagulase positive Staph species 3+, and Malassezia yeast 1+. A flush solution of the composition was continued for two weeks and the ear was reexamined. The external auditory canals were wide open and showed no purulent debris or inflammation indicative of any previous infection. General cleaning on a regular basis was recommended with Epi-Otic®.

After 1 month, Lillie returned with a second bout of otitis occurring post grooming. It was learned that the groomer had been applying a medication in the ears for “ear mites” and the infection started within 48 hours post application. The owner was again instructed to flush the ears with the composition comprising Trihydroxy solution as described herein including a steroid and have them rechecked in 1 week. Within 1 week the owner called back to report that the ears had cleared up and that she would not need the recheck appointment.

Lillie continued to be seen for underlying allergies and has been itching at her ears causing sores and lesions on the external pinna. Otic cytology revealed no etiologic agent, just inflammatory and epithelial cells. Although the generalized erythema has become more prevalent on Lillie's body, her ears have remained infection free during observation with weekly routine cleansing and flushing with the composition solution.

### Example 3

A clinic owned, 5-year-old yellow lab (Seger) was seen in a veterinary hospital in December of 2014 and was diagnosed with chronic bilateral otitis. He was treated in the past at other veterinarians' offices with regular ear cleansers, Zymox™, Epi-otic™, and Triz EDTA™. Multiple antibiotics and formulations that contain topical antibiotics, such as gentamicin, nystatin and/or clotrimazole (Otomax™, Mometamax™, or Panalog™) were utilized along with cleaning, but with reoccurrence of the bilateral otitis happening within several weeks after cessation of meds. Small amounts of steroids for inflammation were also utilized as was BNT weekly packing containing enrofloxacin, nystatin, and triamcinolone. Cultures initially produced results that included mixed infections of cocci/rod bacteria as well as 2-3+yeast.

In March 2015, Seger was cultured again and was diagnosed with a 4+Pseudomonas Aeruginosa infection in both ears along with a 1+yeast isolate. Seger's owner was instructed to flush ears daily with the disclosed composition including dextrose as a nutrient and recheck in two weeks. Upon recheck, the purulent discharge was nearly gone and aural skin ulcerations were cleared up significantly.

Seger was seen again two weeks later (4 weeks after initially starting treatment) and all of the otitis had cleared up and no sores or discharge were noted in either canal. The owner was instructed to use the disclosed composition as a routine cleanser/treatment weekly thereafter. As the owner ran out of the disclosed composition, Seger was seen again in October 2015 for otitis. No complications were seen during treatment.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method for treating pathogenic microbial infections of animals and humans, the method comprising:
topically applying to wounds and/or infected tissue a composition comprising a liquid preparation comprising 3-(trihydroxysilyl) propyldimethyloctadecyl ammonium chloride at a concentration in a range of from about 0.01 w/v % to about 25.0 w/v % in water; and
repeating the topical application of the composition at a dose and frequency effective to treat the pathogenic microbial infections having a negative cell wall charge, wherein the negative cell wall charge is ionically attracted to a positive charge of the composition causing microbial cell death.

2. The method of claim 1, wherein the pathogenic microbial infections are of skin, subcutaneous tissues, cornea and/or conjunctiva.

3. The method of claim 1, wherein the 3-(trihydroxysilyl) propyldimethyloctadecyl ammonium chloride is formed by reaction of 3-(trimethoxysilyl) propyldimethyloctadecyl ammonium chloride and water.

4. The method of claim 1, wherein the liquid preparation further comprises propylene glycol.

5. The method of claim 1, wherein the composition further comprises dimethyl sulfoxide.

6. The method of claim 1, wherein the composition is encapsulated within microcapsules.

7. The method of claim 1, wherein the composition is coated on an outer surface of microcapsules

8. The method of claim 1, wherein the composition is encapsulated within microcapsules and coated on an outer surface of the microcapsules.

9. The method of claim 1, wherein the wounds and/or infected tissue comprise a biofilm having a biofilm matrix.

10. The method of claim 1, wherein the composition further comprises an adjuvant comprising a proteolytic hydrolase enzyme.

11. The method of claim 10, wherein the enzyme is N-acyl homoserine lactone.

12. The method of claim 10, wherein the enzyme is a cellulase.

13. The method of claim 1, wherein the composition further comprises a nutrient.

14. The method of claim 13, wherein the nutrient is a sugar.

15. The method of claim 14 wherein the sugar is selected from the group consisting of monosaccharides, disaccharides, polyols and/or mixtures thereof.

16. The method of claim 15, wherein the sugar is a monosaccharide.

17. The method of claim 16, wherein the monosaccharide is dextrose.

18. The method of claim 1, wherein the composition further comprises an anti-inflammatory.

19. The method of claim 18, wherein the anti-inflammatory comprises a steroid.

20. The method of claim 18, wherein the anti-inflammatory is selected from the group consisting of hydrocortisone, betamethasone, dexamethasone and/or mixtures thereof.
